(19) United States
(12) Patent Application Publication
(10) Pub. No.: US 2009/0272029 A1
(43) Pub. Date: Nov. 5, 2009

(54) Methods for Treating Live Plants or Live Plant Parts or Mushrooms with UV-C Light

(75) Inventors: Arne Aiking, Almen (NL); Frank Verheijen, Wageningen (NL)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Clean Light, Wageningen (NL)

(21) Appl. No.: 12/083,994

(22) PCT Filed: Jul. 21, 2006
(86) PCT No.: PCT/NL2006/050188

§ 371 Date: Apr. 23, 2008

(30) Foreign Application Priority Data
Oct. 24, 2005 (EP) 05109881.2

(51) Int. Cl. A01G 7/00; A01G 1/00; A01G 9/20; A01B 76/00

(52) U.S. Cl. 47/1.43; 47/58.1 LS; 47/58.1 FV; 47/17; 47/1.3

(57) ABSTRACT

The present invention relates to a method for controlling pathogen growth on live plants and mushrooms using UV-C light and an apparatus for use in the method. Also provided are methods for removing surplus leaves and methods for destroying aerial plant parts prior to harvest of underground roots, tubers or bulbs.

### FIELD OF THE INVENTION

The present invention relates generally to agricultural production methods, and more specifically to the reduction or elimination of damage caused by plant pathogens such as Botrytis, Phytophthora and others, on living plants or mushrooms. Pathogen growth on living plants, or plant parts, or mushrooms is controlled using UV-C light, without having a negative effect on the growth, development and yield of the plants or mushrooms. The invention further relates to an apparatus for controlling the growth of pathogenic microorganism. Also provided is a method for removing surplus leaves from live plants using UV-C light and for destroying aerial plant tissues of underground crops prior to harvest.

### BACKGROUND OF THE INVENTION

The effective protection of agricultural crops against infection and damage caused by pathogenic microorganisms has long been a troublesome area of agriculture. In particular, infection by plant pathogenic fungi, such as those in the genus Botrytis or Phytophthora, can result in severe yield loses due to the damage caused on valuable agricultural crops outdoors, and even more so in greenhouse settings.

Most growers treat attacks by fungi with fungicides. These carry a price tag in terms of procurement, but also in terms of labor required to apply the fungicide. In addition, the concerns in the public domain on the long-term effects of fungicide use on the environment and on human health are increasing.

In the case of greenhouse or tunnel grown crops, pathogen attacks can be particularly troublesome, because the higher relative humidity and generous growing conditions of a controlled environment facilitates the growth of not only plants, but also of many pathogens. Growers are, thus, effectively forced to lower the relative humidity of their greenhouses or tunnels, by venting more, which in many cases may increase their heating costs, thus adding significantly to the total cost of chemical pathogen control.

It has been known for some time that UV light can have fungicidal effects. Alert greenhouse growers have observed that the absence of UV (such as underneath large plants, or under a greenhouse/tunnel covers such as glass, polyethylene or other materials that inhibit the transmission of UV light, which is normally present in sunlight) can increase the presence of fungal growth on an agricultural crop.

UV light can be divided into different classes based on wavelength, including ultraviolet A (UV-A) at about 350 nm, ultraviolet B (UV-B) at about 300 nm and ultraviolet C (UV-C) at about 250 nm. Not unexpectedly, the effectiveness of UV light in producing biological changes can differ at different wavelengths.

For fungal treatment, the use of UV light is attractive in that it is a non-chemical treatment that leaves no toxic residue on the crop or in the environment. It has been demonstrated that UV light can inactivate fungal growth. However, UV-A and UV-B have been shown to cause damage to human skin end human eyes. Furthermore, UV-A and UV-B have been demonstrated to be carcinogenic, whereas UV-C is reportedly not carcinogenic.

Up to date, UV-C light has been used to disinfect water or surfaces or to treat post-harvest plant material, such as harvested fruit and vegetables, which are removed from the living/growing/photosynthesizing plant. For example Marquenie et al. (2002, Int.l Food Microbiol 74: 27-35) used UV-C (254 nm) tested the effect of UV-C and/or heat treatment on the viability of conidia of the post-harvested pathogens Botrytis cinerea and Monilinia fructigena. Such treatment is useful for reducing post-harvest damage caused by pathogens during long term storage and transport of harvested fruit and vegetables.

EP0007459 describes the use of UV light having a broad wavelength (200-400 nm) in high doses of 2-300 m W/m², wherein the lower level still corresponds to 0.17 J/cm². Neither the use of UV-C light as such (without substantial amounts of other UV light such as UV-A and/or B) nor the use of lower dosages is suggested. In addition the examples are purely theoretical.

WO2004/089075 describes a method for controlling microorganisms using UV-C and ozonized water, applying dipole electric air jet technology and wetting agents. Thus, two antimicrobial agents are combined, which are apparently useful in the field to combat mixed infections and insects. The technique is only suitable for field grown plants. There is no indication that UV-C may be used as such or which dosages may be effective.

### SUMMARY OF THE INVENTION

The present invention seeks to provide a non-chemical, non carcinogenic treatment of pathogen growth on living plants which affects the pathogen without causing any permanent negative effect on the crop plant, in particular without having a negative effect on the normal growth and development of the plant.

It is an object of the present invention to provide an anti-pathogenic treatment method that allows greenhouse/tunnel growers to accept higher relative humidity it their greenhouses/tunnels. Because pathogen growth is controlled without affecting the normal growth and development of the plants, the crop yield is significantly increased (as yield losses caused by pathogens are reduced). Thus, a superior crop yield and/or significantly lower heating costs are the result of the method.

It is a further object to provide such a treatment without undesirable side effects, such as the carcinogenic effect of UV-A and UV-B light.

It is also a further object of the present invention to provide an anti-pathogenic treatment method that is effective, even when a certain pathogen population may have developed resistance to chemicals.

It is also an object to provide a treatment for controlling pathogen growth in greenhouses/tunnels that is so effective that growers can allow the relative humidity in their greenhouses/tunnels to increase, thus allowing their crop to grow more efficiently, and reduce the energy consumption substantially.

It is also an object of this invention to provide treatment for controlling pathogens on agricultural crops that are being harvested within the next few days. Many fungicides carry a Pre Harvest Interval (PHI) of three days or longer, so that fungus control using such chemicals becomes impossible.

It is a further object of the invention to provide an apparatus for controlling, especially reducing, pathogen growth on a plant, or at least a part thereof, for use in a method according to the invention.

According to the invention an apparatus for controlling pathogen growth on a plant (or at least a part thereof) for use in a method according to the invention, comprises

a light source of UV-C light; wherein the light source emits essentially no UV-A and UV-B light; but at least 90%, 95%, 98%, 99% or more of only UV-C light;
optionally the light source further comprises a quartz tube or casing around it, so that UV-C emission is not reduced and dust and dirt does not collect on the light source itself but on the quartz tube; the dust and dirt can be easily removed by e.g. using high pressure sprayers (spraying e.g. water);
optionally the quartz tube may further comprise a Teflon layer on the inside and/or outside, so that breakage or damage of the quartz tube does not result in particles scattering; essentially all broken particles remain attached to one another by the Teflon layer and the light source can be replaced easily;
transportation means for passing the light source by the plant (or at least a plant part), wherein during one pass of the plant by the light source the plant (or plant part) is treated with an amount of UV-C light which is high enough to reduce (or prevent) plant tissue damage caused by said pathogens but which is low enough not to damage permanently said plant. In one embodiment, the UV-C light is high enough to control (especially reduce) the pathogen growth, while at the same time it does not have a negative effect on the growth, development and/or yield of the plant.

By passing the light source by a plant or the plant by a light source the plant (or plant part) will be exposed for a predetermined limited time. In this limited time the pathogen growth will be controlled, especially reduced. Consequently, the overall amount of pathogen biomass and infection by pathogens is decreased, giving the plant time to recover from the infection. This recovery enables the plant to grow healthier, resulting in superior crop yield.

In a further embodiment of the invention the amount of UV-C light is between 0.002 (or 0.0025) and 0.16 J/cm² during a period of 24 hours, more preferably between 0.002 (or 0.0025) and 0.15 J/cm², especially equal to or below 0.16 or 0.15 J/cm².

It has been found that a fluence in this range in a tissue of a plant is suitable to control the pathogens and that surprisingly only very low UV-C dosages are required to achieve and effective control. The optimal value of fluence depends on the plant species, the growth stage, type of pathogen and growth stage of the pathogen.

### Definitions

“UV-C light” or “UV-C radiation” refers to ultraviolet light (or radiation) having a wavelength of between 240 and 260 nm. UV-C light, having a wavelength of between 243 and 255 nm is preferred; in some embodiments, a wavelength of between about 245 and 247 nm is particularly preferred, as it has been observed that the anti-pathogenic effect of UV-C light tends to peak at this wavelength range. This definition encompasses wavelengths of 240-260 nm, as well as the end-point values as such or values or ranges in between the end-points, such as about 254 nm or about 260, 261, 262, 263, 264 or 265 nm.

“Live plants” or “living plants” is used herein to refer to plants of any growth stage, ranging from seedling stages to mature plants. This term is used to not include harvested plants or severed plant parts (such as seeds, fruit, etc.), with the exception that in one embodiment also “plant cuttings” are included herein, as these cuttings are capable of rooting and will grow into a plant after planting.

“Parts of a plant” refer herein to parts of the live plants, which are not removed from the plants. For example, the stem or lower side of the leaves are parts of a whole plant. Also, the lower 75%, 50%, 25% or 10% of a plant are parts of the plant.

A “plurality of plants” are plants grown in proximity of each other, e.g. side by side in rows or in a field.

“Aerial tissue” or “aerial plant parts” is the plant tissue above ground, especially the foliage, stems, flowers, and developing fruit.

“Mushrooms” include herein all species of (preferably edible, cultivated) mushrooms, such as champignon (Agaricus bisporus), shiitake (Lentinula edodes), oyster mushroom (Pleurotus ostreatus), Boletus species (e.g. B. edulis), Chanterelle (Cantharellus cibarius), etc.

“Live mushrooms” refers to mushrooms at any growth stage, in particular any growth stage of fruiting bodies.

A “plurality of mushrooms” refers to a mushrooms grown in the proximity of each other.

“Pathogen” or “plant pathogen” refers herein to microorganism, such as fungi, bacteria, mycoplasmas and viruses, which are able to cause diseases (e.g. seen as symptoms) on live plants, i.e. on host plants. Especially referred to are pathogens which are present during at least one part of their life-cycle on the exterior surface of one or more of the aerial parts of plants. Also included herein are pathogenic insect and nematode pests.

“Insects” refers herein to any insect species, preferably to plant pests, i.e. insects which damage plants.

“Contact” or “contacting” in the context of UV-C light refers to the shining of the light onto a surface and therefore the exposure of the surface to the UV-C light. “Contacting with” and “exposure to” are herein used interchangeably.

“Controlling pathogen growth” refers to the reduction of the total amount of one or more pathogens on the plant or on one or more plant parts. It is immaterial, whether pathogen amount is reduced due to parts of the pathogen being killed, damaged, or affected in their growth rate, reproduction and/or spread. It also refers to a reduction in pathogen-induced yield loss, as the overall disease pressure (biomass of one or more pathogens) on the plants is reduced.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”, e.g. “a plant” refers also to several plants.

### SHORT DESCRIPTION OF DRAWINGS

The present invention will be discussed in more detail below, using a number of exemplary embodiments, with reference to the attached drawings, in which

FIG. 1 shows a first exemplar embodiment of an apparatus for controlling pathogen growth on a plant (or part thereof) for use in a method according to the invention.

FIG. 2 shows a second exemplar embodiment of an apparatus for controlling pathogen growth on a plant (or part thereof) for use in a method according to the invention.

FIG. 3 shows the effect of UV-C on sporangia germination (%) of Phytophthora infestans.

### DETAILED DESCRIPTION OF THE INVENTION

It was surprisingly found that low levels of UV-C light are highly effective in controlling plant pathogens, whereby the vitality and yield of the plants is increased. Although UV-C light has been used as disinfectant in the past, the effective dosages described were high and application to live plant tissue was only done if the tissue was protected by a thick cuticle covered by wax (such as harvested fruit and vegetables, which do not grow and/or photosynthesize).

The present finding allows for the first time the effective control of pathogens on live, actively growing and/or photosynthesizing plant and/or mushroom tissues. Dosages of 0.16 or 0.15 J/cm² of tissue surface (i.e. 160 or 150 mJ/cm²) or even significantly lower dosages may be used according to the invention. For example, Phytophthora infestans damage can be reduced significantly using as little as 0.002-0.01 J/cm² tissue (2-10 mJ/cm²) applied over a period of 24 hours, with an optimal dosage being about 0.01 J/cm² (10 mJ/cm²).

In addition other applications of UV-C light have been found. For example in one embodiment mushrooms, which are fungi themselves, can be protected against damage caused by other fungal mushroom pathogens. In another embodiment it was found that UV-C light can be used to remove (“burn”) lower leaves of live plants, in such a way that the area where the leave attaches to the stem is not damaged and seals off naturally by forming a protective layer, thereby reducing the incidence of diseases which otherwise (using hand-removal of leaves) would enter the wound.

In a further embodiment a method for destroying above ground aerial parts of plants is provided.

The different embodiments are described in detail herein below.

### Methods for Controlling Plant Pathogens According to the Invention

In one embodiment the present invention provides a method for controlling, especially for significantly reducing, pathogen growth on one or more living plants, especially on a plurality of plants (or on one or more parts thereof, such as the lower half or lower ⅓rd or ¼th of the plant), by contacting at least one or more aerial parts of said plants periodically with UV-C light for a time and at a proximity and intensity sufficient to control one or more pathogens. The UV-C light has especially a negative effect on the pathogen(s), and preferably reduces the amount of pathogens in the area treated. For example, all or part of the fungal mycelium which comes into contact with the UV-C light may be killed, whereby the overall disease pressure on the plurality of plants is reduced. Thus, the pathogens' growth, viability and/or infectivity and/or reproduction may be reduced by the UV-C treatment. Thereby, the yield of the plurality of plants is increased compared to control plants which were not treated in the same way (provided that the initial disease pressure to which the plants were exposed was similar). In a preferred embodiment the growth and development of the plant or of the plurality of plants is not affected negatively by the UV-C treatment, and the yield is also not affected negatively, and is most preferably significantly increased compared to control plants.

In one embodiment of the invention the plant tissue exposed to the UV-C light is not damaged (see below), while in another embodiment some plant tissue parts may be damaged by the UV-C light (e.g. the lower leaves exposed to the UV-C may show UV-C induced symptoms or even die-off or “burn”; see further below), while the overall plant growth and yield are not affected negatively (i.e. the plants continue growing normally and the yield is at least identical to, but preferably at least 1%, 2%, 3%, 4%, 5%, 10%, 20%, 30%, 40%, 50%, 60%, or more, higher than for control plants).

In another embodiment the present invention provides a method for significantly reducing pathogen damage (i.e. protecting plants against pathogen damage) of one or more living plants (a plurality of plants), by exposing at least aerial parts of said plants which are sensitive to be infected by pathogens, one or more times (periodically) to UV-C light for a time and at a proximity and intensity sufficient to have an effect on (i.e., control, especially reduce) the pathogen growth (e.g. reducing the viability and/or infectivity and/or reproduction) without damaging the plant tissue.

Especially provided is a method for reducing plant tissue damage caused by one or more plant pathogens, whereby the method comprises exposing live plants (or parts thereof) one or more times with an amount of UV-C light which is high enough to reduce plant tissue damage caused by said pathogen(s) but which is low enough to not result in permanent damage of said plant tissue. Especially, growth and yield of the plants are not affected negatively.

Plant tissue damage refers herein to the visible, macroscopic tissue damage, which can be scored using visual assessment. Two types of tissue damage can be distinguished. The first type of tissue damage is damage caused directly or indirectly by one or more plant pathogens. This damage is seen as typical disease symptoms, such as e.g. leaf spots, stem spots, chlorosis, necrosis, or cankers. The term “damage” also includes the external coverage of tissue with the pathogen(s), such as live or viable fungal mycelium. Each type of pathogen is known to cause a defined set of symptoms on a host species. The second type of tissue damage is damage caused by UV-C treatment, when too high dosages are applied. These symptoms are also visible macroscopically, as e.g. lesions, chlorosis, etc. However, in one embodiment the present invention employs UV-C dosages which do not cause visible damage to the plant, i.e. neither UV-C induced symptoms nor any other effects on growth and development (such as stunting, deformations, etc) are seen.

In particular, it has been found that amounts of UV-C light between 0.002 (or 0.0025) and 0.16 or 0.15 J/cm² during a period of 24 hours enables not to induce any, or at least not to induce plant tissue damage which has a negative effect on growth and yield of the plants, while still having an anti-pathogenic effect, i.e. controlling pathogen growth. Thus, especially, the normal growth and yield of the plurality of plants are not affected negatively, while pathogen growth is controlled. The optimal dosage or dosage range may depend on the plant species or plant tissue/pathogen combination, as will be described further below. The upper dosage limit can, for example, be determined in dose-response experiments, where plants or plant parts of a species (preferably all at the same developmental stage and grown under the same conditions) are exposed to (contacted with) varying amounts of UV-C light and by then choosing the dosage which does either not lead to any visible symptoms or which does at least not have a negative effect on plant growth and yield. When referring to the exposure to UV-C light herein, it is preferred that essentially only UV-C light is contacted with the tissue, i.e. the light source does not emit substantial amounts (i.e. less than 10%, preferably less than 5% or 2%, most preferably less than 1% or preferably 0%) of UV-A and UV-B light.

Thus, the UV-C treatment according to one embodiment of the invention uses a dosage of UV-C, which significantly reduces pathogen-caused damage (direct and/or indirect symptoms) in treated plants compared to control plants (plants not treated with UV-C), while not affecting the growth and yield of the treated plants. Thus, preferably, the typical disease symptom(s) caused by the pathogen(s) is/are significantly reduced, either on the whole plant or on the part(s) exposed to UV-C. A “significant reduction” refers to a reduction of at least 5%, 10%, 15%, 20%, 30%, 50% 60% or more of one or more symptoms compared to control plants (or parts). This reduction can be assayed and quantified by regular visual scoring, or indirectly, by measuring yield of treated plants compared to control plants.

In one embodiment, the reference to a significant reduction in tissue damage caused by the pathogen includes also a significant reduction in growth of the pathogen(s). This can, for example, be measured by assessing the amount of live or viable pathogen structures itself, for example the amount found on the external surface(s) of the aerial plant parts, or the total pathogen biomass found on the plants/plant parts. Thus, in one embodiment a method for reducing the amount of live or viable pathogen(s) on a plant or plant tissue (especially on the external surface of the plant or plant parts) is provided herein. For example, the amount of (live or viable) fungal mycelium and/or (live or viable) fungal reproductive structures, such as spores (e.g. conidio-spores, ascospores, sclerotia, sporangia, zoospores, etc.) is preferably reduced by at least 5%, 10%, 15%, 20%, 25%, 30%, 50% 60% or more (most preferably 100%) on the UV-C treated plants or plant parts, compared to controls.

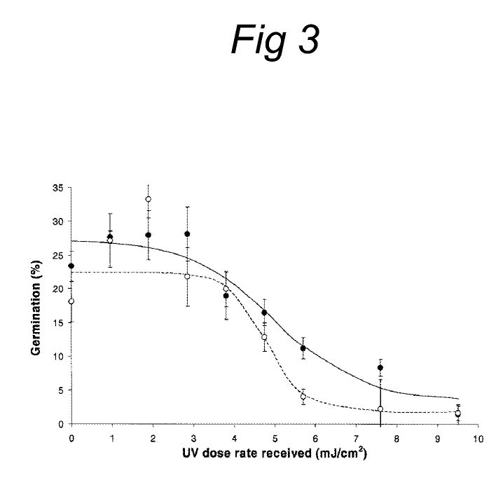

Whether the amount of UV-C dose affects only the viability of the mycelium or whether also the viability of spores or reproductive structures such as sporangia is affected depends on the type of spores and reproductive structures produced by the pathogen. However, for an effective control it is sufficient to significantly reduce the viability of the vegetative structures (mycelium), and an additional reduction of reproductive structures is not necessary although desirable and possible for some pathogens, such as P. infestans. It was surprisingly found that the low dosages UV-C light used were sufficient to not only significantly reduce viability of the mycelium but also to reduce viability (seen as a significant reduction in % germination when using UV-C light of about 6 to 10 mJ/cm²; see also FIG. 3) of the sporangia and/or zoospores of Phytophthora infestans. It is noted that the term “fungal” and “fungus” as used herein encompasses oomyctes such as P. infestans.

The above applies equally to other pathogens, such as bacteria or viruses, which are during at least one part of their life cycle present on one or more external, aerial plant surfaces. A reduction in the pathogen growth itself can be assayed by either assaying symptoms on the plant tissue or by assaying the amount of viable spores or sporangia and/or mycelia, e.g. present on the external surface, at one or more time points compared to control plants/tissues. For example, the presence or absence of the pathogen on a given plant or plant tissue can be assayed and optionally quantified, using for example visual means, molecular methods (e.g. PCR based methods), immunological methods, microscopy methods and/or bioassays.

In the methods, the growth, development and yield of the plants or plurality of plants is not affected negatively. Growth and development of the plants is comparable to that of non-treated control plants and is evaluated visually. Yield can be measured in various ways, e.g. by measuring the weight or size of harvested parts (e.g. average fruit size and/or weight).

The method, therefore, comprises (a) contacting one or more plants or plant parts with a predetermined dosage of UV-C light (one or more times) and optionally further (b) assessing either the tissue damage visually at one or more times and/or optionally (c) assessing the pathogen growth, especially the amount of live or viable pathogen, on the plants or plant parts, and/or (d) assessing the growth and yield of the plants, compared to controls.

Thus, the lower UV-C dosage limit can also be determined in dose-response experiments, whereby the plants or plant parts (again preferably all at the same developmental stage and grown under the same conditions) are exposed to (contacted with) varying dosages of UV-C and one or more pathogens and the development of symptoms and/or of the pathogen growth itself is assayed.

The reduction in plant tissue damage preferably leads to an increase in yield, most preferably by at least 2, 5, 10, 15, 20, 30, 40, 50 or more percent yield compared to the yield of control plants not exposed to (contacted with) UV-C. Also the vitality of the plants is increased, which can be assessed visually.

The way of contact between the plant tissue and the UV-C light can be varied, depending on the plant species/plant tissue-pathogen combination and on the plant architecture. For example, once the optimal dosage for treating a certain tissue of a plant species has been determined, the dosage may be applied as a single dosage or may be divided into two or more dosages, which are applied consecutively within a certain time-interval, e.g. within one or more minutes, hours, or days (e.g. 1, 2, 3, 4 or 5 times per week or more), etc. Additionally or alternatively, the distance between the tissue and the source of UV-C light may be varied, as described herein below.

In one embodiment crop or ornamental plants grown in controlled environments, such as greenhouses or tunnels (e.g. polyethylene tunnels), are contacted with UV-C light, although in another embodiment also field crops or ornamental plants are contacted.

When exposing plants to the UV-C light, the light source(s) may be arranged so that exposure takes place from one or more sides (e.g. two sides, left and right, of a plant or of a row of plants) and/or from the top. Optionally the lights may be arranged on the top and may be lowered into the plants. For example, a tractor may pull a wide boom behind it, wherein the spraying nozzles have been replaced by UV-C lights. The UV-C lights can expose the plants to UV-C light from the top, or they can be lowered into the crop, in such a way that the lights are in between the plants. The plants may be bent over by the lowering of the lights. For example wheat plants or soybean plants are flexible, so that they bend when the boom is lowered and bounce back when the boom has passed. An apparatus capable of lowering the UV-C lights to a position in between the plants is an embodiment of the invention, as described further below.

The plants that can be treated by the present method can be any plants that are susceptible to pathogen, especially fungal, attack and where the pathogenic microorganism is at least partially located on the outside of the plant, i.e. the plant tissue surface. Thus, the plants suitable for treatment with the present invention include plants that are commonly grown in greenhouses or tunnels, such as vegetables, flowers, fruits, and medicinal plants, as well as outdoor crops such as vegetables, forage, cereals, fruit plants, trees or tree seedlings, bulbs/flowers, and medicinal plants. It is also in one embodiment contemplated that the method may be used in conjunction with a method to move plant foliage, and thus expose the pathogen growth to the UV-C light. Such devices may include fans, or physical mobile objects to clear foliage. In one embodiment the UV-C light is advantageously used to cause death and/or chlorosis/necrosis of some plant tissue, especially (lower) leaves, which normally have to be removed by hand (see herein below).

It was also surprisingly found that the amount of insect pests and insect damage found on plants could be reduced by simultaneously contacting insects and plants or plant parts with UV-C light as described above, while beneficial insects such as bees seem not to be affected. The insects (or any developmental stage thereof, such as eggs or larvae) may be present on the plants and/or on the ground under the plant. It is presumed that the UV-C light either defers the insects, or confuses and/or kills the insects (or one or more developmental stages such as eggs and/or larvae and/or mature insects), especially insects which can sense UV-C light, such as centipedes, millipedes, moths, lice etc. It is, therefore, also an embodiment of the invention to reduce insect damage and to reduce yield loss caused by insect pests. The embodiments described for pathogens equally apply to insects.

It was also found that nematodes are killed if exposed to UV-C light as described. This is advantageous in soil-grown crops, where the soil may be contaminated with nematodes. In this embodiment the soil and/or base of the plant and/or mushrooms is exposed to the UV-C dosages one or more times, as described for tissue above.

As mentioned above, any plant species may be used in the method, and preferably vegetable species, field crop species and ornamental plant species are used in the method. These include plants of the following species: maize/corn (Zea species), wheat (Triticum species), barley (e.g. Hordeum vulgare), oat (e.g. Avena sativa), sorghum (Sorghum bicolor), rye (Secale cereale), soybean (Glycine spp, e.g. G. max), cotton (Gossypium species, e.g. G. hirsutum, G. barbadense), Brassica spp. (e.g. B. napus, B. juncea, B. oleracea, B. rapa, etc), sunflower (Helianthus annus), safflower, yam, cassava, tobacco (Nicotiana species), alfalfa (Medicago sativa), rice (Oryza species, e.g. O. sativa indica cultivar-group or japonica cultivar-group), forage grasses, pearl millet (Pennisetum spp. e.g. P. glaucum), hemp (Cannabis sativa), tree species (Pinus, poplar, fir, plantain, Picea, etc.), tea, coffee, oil palm, coconut, vegetable species, such as tomato (Lycopersicon ssp e.g. Lycopersicon esculentum, renamed as Solanum lycopersicum), potato (Solanum tuberosum, other Solanum species), eggplant (Solanum melongena), peppers (Capsicum annuum, Capsicum frutescens), pea, zucchini, beans (e.g. Phaseolus species), cucumber, artichoke, asparagus, broccoli, cabbage, garlic, leek, lettuce, onion, radish, turnip, Brussels sprouts, carrot, cauliflower, chicory, celery, spinach, endive, fennel, beet, fleshy fruit bearing plants (grapes, peaches, plums, strawberry, mango, apple, plum, cherry, apricot, banana, blackberry, blueberry, citrus, kiwi, figs, lemon, lime, nectarines, raspberry, watermelon, orange, grapefruit, etc.), ornamental species (e.g. Rose, Petunia, Chrysanthemum, Lily, Tulip, Gerbera species), herbs (mint, parsley, basil, thyme, etc.), woody trees (e.g. species of Populus, Salix, Quercus, Eucalyptus), fibre species e.g. flax (Linum usitatissimum). Particularly preferred plants and plant parts are potato plants, wheat and other cereals (especially winter wheat), field vegetables such as onions, greenhouse vegetables (tomato, cucumber, sweet pepper, etc.) and fleshous fruit bearing plants, such as fruit trees (apple, pear, plum, etc).

The method is used to significantly reduce or prevent pathogen growth and plant damage caused by one or more pathogens which infect the above species. The pathogen(s) may be fungal species (including oocmycetes), bacterial species or viruses or viroids. In a preferred embodiment the pathogen is a necrotrophic fungus, preferably Botrytis cinerea. In another preferred embodiment the pathogen is a member of the genus Phytophthora, especially P. infestans. The types of pathogens treated with the method include all plant pathogens, especially fungi, commonly found on the exterior of plants during some part of the life-cycle (especially fungi which produce mycelium or reproductive structures on the exterior surface of plant tissues) and that can be exposed in a practical manner to the UV-C light, such as Botrytis on the stems of tomato plants and on other plant species and plant parts, P. infestans on potato or various rust species, such as Asian soy rust on soybean plants or smut species.

Pathogens of tomato, for example, include the following species: Botrytis cinerea Colletotrichum coccodes, Corynebacterium michiganense, Bacterial speck (Pseudomonas syringae), Clavibacter, Xanthomonas campesiris pv vesicatoria or Xanthomonas vesicatoria, Tobacco or tomato mosaic viruses (TobMV, TomMV), Alternaria alternate, Early blight (Alternaria solani), Gray Leaf Spot (Stemphylium solani), Late Blight (Phytophthora infestans), Septoria Leaf Spot (Septoria lycopersici), Cladosporium fulvum, Phytophthora parasitica, Fusarium oxysporum, Sclerotium rolfsii, Pythium and Rhizoctonia, tomato spotted wilt virus (TSWV).

Pathogens of cucumber, for example, include the following species: Botrytis cinerea, Erwinia carotovora, Colletotrichum orbiculare, Phomopsis sclerotioides, Rhizoctonia solani, Pseudoperonospora cubensis, Fusarium oxysporum f. sp. Cucumerinum, Didymella bryoniae, Phoma cucurbitacearum, Cladosporium cucumerinum, Corynespora cassuiicola, Pseudomonas syringae pv. lachrymans, Erwinia tracheiphila, Cucumber Mosaic Virus, Papaya Ringspot Virus (PRSV), Watermelon Mosaic Virus (WMV), Zucchini Yellow Mosaic Virus (ZYMV).

Pathogens of pepper, for example, include the following species: Xanthomonas campestris pv. vesicatoria, Leveillula taurica, Cercospora capsici, Scierotium rolfsii, Rhizoctonia solani, Pythium sp., Phytophthora capsici Cucumber mosaic virus (CMV), tobacco mosaic virus (TMV), tobacco etch virus (TEV), tomato spotted wilt virus (TSWV), alfalfa mosaic virus (AMY), Potato virus Y (PVY), pepper mottle virus (PeMV).

For vegetable species, diseases per crop species can also be found at http://vegetablemdonline.ppath.cornell.edu/Home.htm; for other plant species, pathogens and their symptoms are also well known in the art.

The method according to the invention is preferably used to prevent yield loss (e.g. reduce damage or infection) caused by species of the following genera: Botrytis, Sclerotinia, Pythium, Fusarium, Phytophthora, Alternaria, Cercospora, Erysiphe, Sphaerotheca, Verticillium, Xanthomonas, Pseudomonas, Stemphylium, Septoria, Peronospora, Erwinia, Mycosphaerella, Albugo, Cladosporium, Microdochium, and Colletotrichum, Clavibacter, as well as various fungal rust species (Uredinales), such as Asian soy rust (Phakospora pachyrhizi) and other rusts, such as cereal rusts, or smut species (Ustilaginales).

Preferably, the whole plants or plant parts (e.g. all or part of the stem, upper or lower surface of the leaves, flowers, developing fruit) are exposed to (contacted with) an appropriate dose of UV-C at one or more developmental stages. For example, seeds may be sown in the greenhouse and treatment may already start after emergence of the young seedlings. Alternatively, only more mature plants are treated. The dosage may need to be lower for younger tissue than for older tissue, but the skilled person can easily determine the appropriate dosage and frequency of application. Also tissue type may influence the optimal dosage. A stem may for example tolerate a higher dosage than a young leaf. Routine experimentation can be used to determine the optimal dosage or minimum/maximum dosage range. The dosage may thus be at least about 0.002, 0.0025, 0.005, 0.006, 0.007, 0.008, 0.009, 0.01, 0.02, 0.03, 0.04, 0.05, 0.06, 0.07, 0.08, 0.1, 0.15, 0.16 J/cm² or more, but less than about 0.17, 0.2 or 0.25 J/cm² during a period of 24 hours.

Preferred ranges of UV-C light include therefore 0.002-0.15 J/cm² or 0.16; 0.0025-0.15 or 0.16 J/cm²; 0.002-0.006 J/cm²; 0.002-0.01 J/cm²; 0.0025-0.006 J/cm²; 0.0025-0.01 J/cm². Such specific dosage ranges which apply a low dosage of a narrow wavelength (UV-C light of 240-260 nm or the end-points or any specific value in between this wavelength such as 254 nm, i.e. essentially without wavelengths below 240 and above 260 nm and without ozonized water or ozone production) per cm² to one or more pre-selected parts of the plants are particularly advantageous because they are particularly effective and do neither harm the plants nor harm the environment around the plants, such as humans or animals. In addition energy is saved and the possibility to fully automate the application reduces labor costs.

The method is particularly effective when carried out with a UV-C lamp having an intensity of between 2 and 100 Watts that periodically travels through the crop, with an effective exposure period of between one second and one minute, and a proximity to the pathogen growth of between 2 cm and 200 cm. It has been observed that such application of UV-C light can kill up to 100 percent of the fungal mycelium growth on a plant, thereby enabling the plant to grow better, and produce a superior product. As mentioned above, UV-C light can also kill (or reduce viability of) reproductive structures such as spores and/or sporangia, whereby at least 10, 20, 30, 50, 60, 70 or 80% or more of the reproductive structures are killed or rendered non-viable.

Without limiting the scope of the invention, it is believed that the application of UV-C light to pathogen growth is lethal because of the close similarity of the UV-C wavelength to the maximum absorption rate of DNA (which is about 260 nm). As such, the application of UV-C can cause photochemical changes in the DNA that either cause immediate death or impair the reproduction of the organism. Because the reproductive cycle of most microorganisms is much faster than that of normal cells, they are much more susceptible to the harmful effect of the UV-C than the cells of the plant.

In another embodiment the use of UV-C light for the control of one or more plant pathogens on live plants or plant parts (especially for the reduction in the amount of one or more pathogens) is provided, whereby plant growth and yield is not negatively affected. Preferably, yield of UV-C treated plants is increased, as described elsewhere herein.

### Methods for Controlling Mushroom Pathogens

The above methods for controlling plant pathogens also apply for the control of mushroom pathogens. It was surprisingly found that mushrooms can be protected from pathogens by applying one or more dosages of UV-C light to the mushrooms, especially to all or part of the fruiting bodies (e.g. the cap and/or the stalk and/or gills or lamella) at one or more time points.

The method as described above for plants can thus equally be applied to mushrooms, such as cultivated mushrooms, for example basidiomycetes or ascomycetes. Especially use for controlling pathogens of the following species is encompassed herein: Agaricus bisporus, Lentinula edodes, Pleurotus spp., Auricularia spp. Volvariella volvacea, Flammulina velutipes, Tremella fuciformis, Hypsizygus marmoreus, Pholiota nameko, Grifola frondosa, and others.

The method and time of application of the UV-C light depends on the cultivation method of the mushroom. Agaricus bisporus, for example, is generally grown in trays, while shiitake is grown in natural or synthetic logs. The Asian paddy-straw mushroom Volvariella volvacea on the other hand is grown in beds of damp rice-straw outdoors. The light source may, therefore, be applied from the top and/or from the sides at one or more time-points. The fruiting bodies of Agaricus bisporus for example begin appearing about 6 weeks after spawning and continue appearing in flushes about 7-10 days apart for the next 6-8 weeks. The UV-C light may thus be applied before and/or during fruiting body appearance.

Pathogens of mushrooms include primarily fungi, bacteria, viruses and insects. Fungal pathogens include for example species of the following genera:

| | Common disease | |
|---|---|---|
| Genus | name | Disease symptoms |
| Dactylium | Cobweb, mildew | White to pink cobweb-like fluffy |
| | | mould. |
| Diehlomyces | Calves brains/ | A competing fungus which produces |
| | false truffle | brain-shaped fruiting bodies. |
| Fusarium | Damping off | Mushrooms wither. |
| Mycogone | Wet bubble/ | Dense white growth on gills. |
| | white mould | |
| Papulaspora | Brown plaster | Brown plaster-like patches on casing. |
| | mould | |
| Scopulariopsis | White plaster | White plaster-like patches on casing. |
| | mould | |
| Trichoderma | Green mould | Dark green mould patches on casing |
| | | spreading to lesions on stems. |
| Verticillium | Dry bubble/ | Brown irregular pitted areas on |
| (V. fungicola) | brown spot | stems and caps. Distortion and |
| | | splitting. |

Bacterial pathogens include Pseudomonas species and viral pathogens include for example MVX (mushroom virus X). Insect pests include a variety of small fly and midge species.

Especially the effective UV-C control of fungal pathogens is surprising, as mushrooms are fungi themselves. The method can be applied without damaging the mushrooms themselves, thereby increasing yield (reducing yield losses) significantly and increasing quality of the mushroom products (especially fruiting bodies produced for the fresh market).

### Methods for Controlling Pathogens on Plant “Cuttings”

In a further embodiment the live plant parts are ‘cuttings’ which are used to clonally propagate plants, such as stem cuttings of herbaceous and woody species (softwood, semi-hardwood or hardwood). Roses, Chrysanthemums and Dahlias are for example propagated using cuttings. Examples of plants propagated by cuttings at the hardwood stage include forsythia, privet, fig, grape, and spirea. The cut stem (or shoot) pieces are generally freed from any attached leaves, leaving open wounds. Off course one or two ends of the cutting also have an open wound.

The embodiments described above for plants thus equally apply for the treatment of cuttings. Suitably, cuttings are contacted with a suitable dosage of UV-C light at one or more timepoints after they are removed from the stock (parent) plant and before they are planted into soil or a suitable growth or rooting medium. The treatment may also be applied at one or more timepoints after the cutting is placed into soil or a suitable growth or rooting medium. The rooting time varies depending on the species. Especially, contact before and/or during rooting and/or optionally even thereafter, during further growth, is suitable for controlling pathogen damage and/or for reducing loss of viability of the cutting. Also the rooting success (% of cuttings which successfully form roots and can develop into mature plants) can be increased significantly using UV-C light, preferably by at least 5%, 10%, 20%, or more, compared to non-treated cuttings.

Thus, in one embodiment the whole cutting, and/or the aerial part of the cutting (after placement into a suitable medium or after transplantation to other medium or into the field) is contacted one or more times with UV-C light of the dosages described above.

### Methods for Removing Surplus Plant Parts, Especially Leaves, and Increasing Plant Vitality

In yet another embodiment of the invention a method for removing surplus plant tissue, especially lower leaves, is provided. This method is particularly suited for greenhouse/tunnel grown plants. The method has significant advantageous over the current manual removal of leaves. Manual removal of lower leaves is carried out because the lower leaves are a source of disease and lower a plants vitality and growth. In addition, old lower leaves inhibit air circulation in greenhouses and tunnels, and block light. Manual removal of lower leaves is therefore commonly done about once a week, e.g. in tomato plants, cucumber or pepper plants. Additionally, the detached leaves have to be removed from the vicinity of the plants as otherwise they provide a source of pathogens.

It was found that the application of a suitable dosage of UV-C light to essentially only the lower leaves results in the leaves becoming brown and dry and falling off the plant in a matter of days. Thereby no open wound is left, as a protective layer is formed in the abscission zone. The plants are therefore not wounded and the disease incidence is reduced. In addition, the dropped off leaves can be easily removed (manually or using fans or other physical devices) and because the dropped off leaves are not fresh and green their potential to form a reservoir for pathogens is decreased. In certain embodiments they may thus not even need to be removed at all.

The method comprises the same steps as already described above, whereby a suitable dosage of UV-C light is applied to the lower leaves of the plants at one or more time points, until the leaves turn brown and dry and preferably until they fall off the stem by themselves. Preferred UV-C dosages are described above, and may be determined using routine experimentation. For example about 0.05 J per cm² is applied during a 24 hour period, and optionally this is repeated several times.

Although preferably low dosages or dosage ranges are applied (as described above), one may optionally in this embodiment also use higher dosages, such as 0.2, 0.25 J/cm² or even higher dosages such as 0.3, 0.4, 0.5, 0.7, 0.8, 1.0, 1.5, 2.0 J/cm² or more.

This method saves labor costs and increases the vitality of the plant by inducing the leaves to abscise ‘naturally’ and by significantly reducing pathogen infection. The plant yield or growth is therefore not influenced negatively but positively.

### Methods for Destroying Above Ground, Aerial Plant Parts During Pre-Harvest

Prior to harvesting underground crops (edible storage organs), such as tubers (potatoes), roots or bulbs, it is common practice to kill the aerial parts by chemical (phytotoxic) spraying. The use of chemicals is not desired. The present invention provides an environmentally friendly method to remove aerial plant parts prior to the harvest of underground crops using U-C light. Optionally the exposure to UV-C light may be combined with chemicals, so that the amount of chemicals is reduced.

It was found that exposure of the entire aerial plant part to one or more suitable dosages of UV-C light is very effective in destroying the aerial tissue in a quick and clean manner, whereby the tissue becomes dry and brownish (and not a suitable source for pathogens). The UV-C dosage is preferably applied one or more times during the one, two or three weeks prior to harvest date. Once the tissue has turned dry and brown, it is easily removed from the field. The removal is much easier than for chemically treated plant parts and can be carried out using the same machinery.

Although preferably low dosages or dosage ranges are applied (as described above), one may optionally in this embodiment also use higher dosages, such as 0.2, 0.25 J/cm² or even higher dosages such as 0.3, 0.4, 0.5, 0.7, 0.8, 1.0, 1.5, 2.0 J/cm² or more.

### Apparatus According to the Invention

The above method is preferably automated, and the contact between the tissue(s) and UV-C is preferably brought about by using an apparatus, comprising a source of UV-C emission and a means for controlling the amount and duration of emission, as well as the distance between the tissue and the UV-C source.

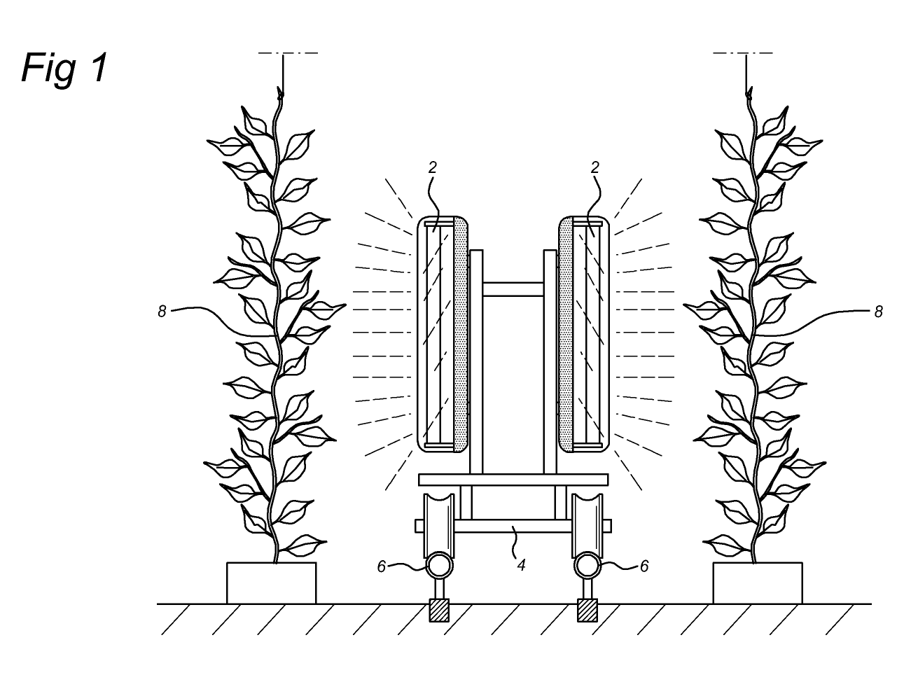

FIG. 1 shows a first exemplar embodiment of an apparatus for reducing pathogens growth on a plant for use in a method as described above. The apparatus comprises at least one light source of UV-C light 2. The light source 2 could be any commercially available UV-C light source which enables to produce an amount of UV-C light between 0.0025 and 0.25 J/cm² during a period of 24 hours or any of the above specified dosages or dosage ranges, e.g. 0.02-0.15 J/cm² during a period of 24 hours. Preferably, the desired UV-C dosage is emitted during a single pass of the light source(s), i.e. preferably e.g. 0.02-0.15 J/cm² of UV-C (or any of the other dosages described in the embodiments of the invention) is emitted during a single pass. The UV-C light applied to the pathogen (e.g. fungus), plants, plant parts or mushrooms is typically supplied by a UV-C germicidal lamp, although other UV-C light sources may also be suitable. A germicidal UV-C lamp is generally of the configuration of a small fluorescent lamp, and requires the same type of peripheral or auxiliary equipment. A UV-C lamp typically contains no phosphorous, but has a drop of liquid mercury dispersed in an argon gas vacuum. The mercury floats within the argon; when electricity is introduced, the mercury atoms discharge IV-C light at approximately 260 nm. The UV-C lamp may include a special glass bulb, cover or lens that allows transmission of most of the UV-C light generated by the mercury arc (up to 74 percent of the UV-C energy can be transmitted through the glass). The intensity of the light source 2 can be increased by placing more then one UV-C lamp next to each other. Preferably, the UV-C light source emits essentially no UV-A and UV-B light. For example one or more low pressure mercury discharge lamp emitting essentially only UV-C light, preferably of a narrow or specific wavelength (e.g. essentially only 254 nm or only 265 nm), may be used.

In a preferred embodiment the UV-C light source is surrounded (preferably entirely) by a quartz shield or tube, which allows the UV-C light to pass through. This embodiment is particularly preferred for use in dusty or dirty environments, such as the field or greenhouses/tunnels, allowing easy cleaning of the light source.

In another embodiment the quartz tube comprises a Teflon layer, either on the inside (near the light source) or preferably on the outside. In case of damage of the quartz tube or of the light source, the Teflon-quartz tube ensures that the no contamination of the environment occurs and allows easy replacement of the light source.

The apparatus further comprises transportation means 4 for passing the light source by the plant, plant parts or mushrooms, wherein preferably during one single pass of the plant, plant part or mushrooms by the light source the plant, plant part or mushroom is treated with an amount of UV-C light which is enough to achieve the desired effect (as described), e.g. enough to control pathogens growth on at least a part of a plant and which does not influence the plant growth or yield negatively. The term yield is meant the crop of a plant or the economic value of a pot plant, trees, flowers or the like. In FIG. 1 the transportation means 4 is a trolley. Heating pipes 6 in a greenhouse or tunnel could function as rails for the trolley. The trolley could include an engine for moving the trolley. If usable heating pipes are available at the top of a greenhouse, the transportation means wilt hang to move the light source along the plants. The transportation means 4 may be any other suitable means of transport, such as a conveyor belt or automatic navigable vehicle, which may include sensors to enable navigation along the plants, and also a tractor or other vehicle which enables movement. In certain embodiments of the invention, the UV-C source may also be stationary (e.g. without transportation means) and the application of the UV-C light is controlled by varying the position and the time of application (e.g. using an on/off switch).

The light source 2 is mounted on the trolley at a position such that at least the area which is to be treated, e.g. the area of the plants which is sensitive for infection with pathogens will be contacted. In case of tomatoes this may for example be the stem in a well known specific range above the ground. Furthermore, the distance between the light source and the plant, plant part or mushroom is such that the tissue of the plant, plant part or mushroom is not damaged permanently by the UV-C light, with the exception of the embodiment above wherein surplus leaves are to be removed by permanent damage of the surplus leaves only and with the exception of destroying aerial plant parts above. The fluence (J/cm²) of UV-C light contacted with the tissue is dependent on the intensity (W/cm²) of the light source, the relative speed (cm/s) between light source and the distance between the light source and the plant (cm). A suitable speed to be applied in a greenhouse or tunnel or outdoors is in the range of 0.01-1 m/s, but other speeds may also be used as long as the desired dosage reaches the desired tissue.

If the apparatus is used to reduce plant or mushroom damage by insects and/or nematodes, the light source should be mounted such that the living area of the insects or nematodes is exposed to UV-C light. For insects or nematodes living on the ground, a special light source could be mounted. A reflector, screen or the like could be used to direct the light to the ground and not to the plant or mushroom. This enables to give the ground a higher dosage of UV-C light without affecting the plant or mushroom negatively, and consequently increase the reduction of insect damage. Other well known light systems are available to distribute the UV-C light to different areas with different intensities.

To obtain an optimal and controllable fluence the apparatus includes a control unit for controlling the fluence. The enable controlling of the fluence the control unit could control the intensity of the light source 2, the distance between area of a plant 8 or plant part or mushroom and the light source 2 or the relative speed of the light source with respect of the area of the plant, plant part or mushroom to be contacted.

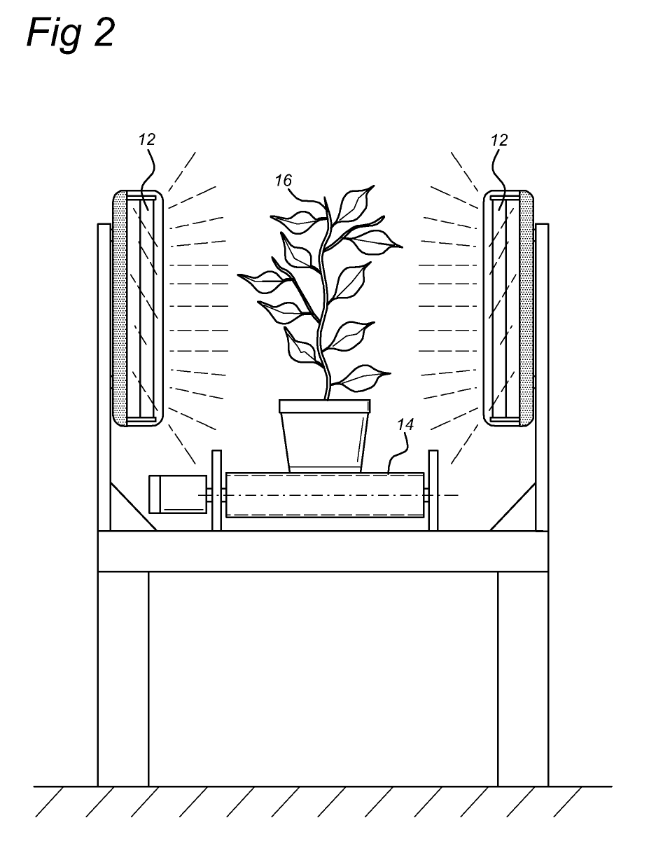

FIG. 2 shows a second exemplar embodiment of an apparatus for reducing pathogens growth on a plant, plant part or mushroom for use in a method as described above. The apparatus comprises at least one light source of UV-C light 12. The apparatus further includes a conveyer belt 14 for passing a plant or mushroom (e.g. in trays or logs) along the at least one lamp. In FIG. 2, at each side of the conveyer belt 14 a light source 12 is positioned. If suitable a light source could be placed above the conveyer belt 14. In stead of a conveyer belt 14 any suitable transportation means could be used to pass plants or mushrooms by the light source.

The light sources 12 are positioned at a position such that the distance between the light sources and the plant 16 is such that the tissue of the plant or mushroom is not damaged permanently by the UV-C light.

To obtain an optimal and controllable fluence on the tissue of the plant or mushroom, the apparatus includes a control unit for controlling the fluence. To enable controlling of the fluence the control unit could control the intensity of the light sources 12, the distance between the area of a plant 16 or mushroom and the light source 12 or the relative speed of the light source 12 with respect of the area of the plant/mushroom to be shined.

The apparatus should be suitable to apply a predefined dosage of UV-C to the desired plant or mushroom tissue, e.g. to the stem, the upper leaf surface or the lower leaf surface or the upper or lower side of the cap or stalk of mushrooms. The design of the apparatus depends to some extent on the growth characteristics of the plant/mushroom species and the production system (field or greenhouse, trays, logs, etc.).

Because a high dose of UV-C light may damage the plant or mushroom, and because low dosages may not damage the pathogen (e.g. fungal) growth sufficiently, it is desirable to have automated control of the light source, so that intensity, application time, and distance from the pathogen (e.g. fungal) growth can be accurately controlled. Therefore, it may be desirable to mount the lights on a carriage that travels through the crop, above the crop or between plant rows or mushroom rows or trays at a predetermined speed depending on the crop to be treated. A particularly suitable speed for tomatoes and green peppers in greenhouses in e.g. the Netherlands may be between 5 and 50 meter per minute. This allows the machine to operate before and after regular working hours, and yet not disturb the circadian rhythm of the plants, and not interfere with normal operations in the greenhouse, while treating every plant once per week, or once a day as may be determined by the grower, in a typical greenhouse.

During the application of UV-C light to the plant or mushroom, the UV-C lamp is preferably positioned sufficiently close to have fungicidal or anti-pathogenic effect (affecting growth, reproduction, infection and/or spread), and yet not so close as to damage the plant or mushroom (except where surplus leaves are to be removed, see above). This position is typically between 2 cm and 200 cm from the plant or mushroom, and distances of 5, 10, 20, 30, 40, 50, 100 cm are envisaged.

Application of the UV-C light should be sufficient to be effective, and yet not too long so as to cause damage to the plant or mushroom. Typically, the duration of light may be between one second and one minute. This consequently defines the minimum and maximum speed of passing by. Obviously, more than one UV-C light source may be used, such as 2, 3, 4, 5, 6, 8, 10, 16, 20 ore more, preferably such that the UV-C dosage desired is provided in a single pass. For example if the light sources are attached to a boom attached to a tractor or other movable device, one or more rows of light sources may be present. For a smaller apparatus such as shown in FIG. 1 or 2, more than one UV-C light may be present on either side, such as 3 lights either side.

Obviously, the UV-C lamp energy, distance and duration of emission (and speed and position of the apparatus) determine the total dosage (J/cm²) brought into contact with the plant or mushroom tissue.

The apparatus could further comprise a fan (not shown) to move the leafs of a plant to enable to treat the stem or other areas of the plant more effectively.

### EXAMPLES

### Example 1

### Control of Botrytis on Tomato Plants

Tomato plants are grown in rows in a greenhouse. An apparatus comprising two UV-C lamps, one on either side of the front end of the apparatus is placed onto rails (e.g. heating pipes) between some of the rows, at a height which brings the UV-C light into contact with about ⅔ of the stem.

The amount of Botrytis cinerea mycelium present on the surface of the stems is assessed at regular intervals, both in the UV-C treated plants and in the control plants. The assessment enables to find the optimal UV-C dosage to damage the Botrytis and to improve the productivity of the treated plant.

By treating a tomato periodically, the Botrytis growth is reduced, which postpones or prevents the instant that the Botrytis completely surrounds the stem of the tomato and thus increases the tomatoes duration of life and yield.

### Example 2

### Reducing Germination of Sporangia of Phytophthora infestans

The effect of UV-C dose rate on the germination of P. infestans sporangia was assessed on water agar. P. infestans sporangia were plated on 1% water agar and exposed to different dosages of UV-C. Germination was determined for 100 sporangia per plate. Four replicates were included for each dose rate.

The results are shown in FIG. 3. Data points represent averages of four replicate plates. The experiment was replicated. Solid dots represent data from experiment 1 and open dots represent data from experiment 2. Error bars represent standard deviation.

The results show that the viability of P. infestans reproductive structures can be significantly reduced using UV-C light. The percentage of germination was reduced by at least 80% using about 6-10 mJ/cm² UV-C.

The foregoing detailed description of the invention has been presented for purposes of illustration and description. It is not intended to be exhaustive or to limit the invention to the precise form disclosed, and obviously many modifications and variations are possible in light of the above teaching. For example for treating little trees planted in rows in open field, the apparatus could span more than one row. In that case the light sources are positioned such that in each row between two rows of trees a light source will be present, so as to enable to treat more than one row simultaneously. The described embodiments were chosen in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

1-18. (canceled)

19. A method for reducing the total amount of one or more pathogens on a living plant, said method comprising contacting a live plant or mushroom, or a part thereof, with a dosage of UV-C, wherein during a period of 24 hours, the dosage of UV-C is about 0.002 to about 0.15 J/cm² of tissue.

20. The method according claim 19, wherein the growth of one or more pathogens is reduced by at least 5% compared to an equivalent control that has not been contacted with said dosage of UV-C.

21. The method according to claim 19, wherein said one or more pathogens comprises one or more of a fungus, oomycete, bacterium, insect, nematode, mycoplasma, or virus.

22. The method according to claim 21, wherein said one or more pathogens comprise one or more of: Botrytis, Sclerotinia, Pythium, Fusarium, Phytophthora, Alternaria, Cercospora, Erysiphe, Sphaerotheca, Verticillium, tobacco mosaic virus, Xanthomonas, Pseudomonas, Stemphylium, Septoria, Peronospora, Erwinia, Mycosphaerella, Albugo, Cladosporium, Microdochium, Colletotrichum, or Clavibacter.

23. The method according to claim 19, wherein the plant is a vegetable plant species, a fruit-bearing species, a field crop species, or an ornamental plant species, or wherein said mushroom is a cultivated, edible mushroom species.

24. The method according to claim 19, wherein the plant is grown in a greenhouse, a tunnel, or in a field.

25. The method according to claim 19, wherein said contacting step is carried out during more than one developmental stage of said plant or mushroom.

26. An apparatus for reducing the total amount of one or more pathogens on a living plant or mushroom, or part thereof, said apparatus comprising
a light source comprising one or more germicidal UV-C lamps;
a cover that allows transmission of most of the UV-C generated by the lamp; and
a control unit for controlling the fluence of UV-C in J/cm², wherein the dosage of UV-C is between 0.002 and 0.15 J/cm².

27. The apparatus according to claim 26, wherein said cover comprises a quartz tube or shield at least partially covering the light source.

28. The apparatus according to claim 26 wherein said control unit controls the intensity of the light source, the distance between the light source and the living plant, mushroom, or part thereof, or any combination thereof.

29. The apparatus according to claim 26, further comprising a Teflon layer outside of the cover.

30. The apparatus according to claim 26, further comprising transportation means.

31. The apparatus according to claim 30, wherein the transportation means is arranged to convey the plant or mushroom, or part thereof, past the light source so as to be exposed to the one or more germicidal UV-C lamps.

32. The apparatus according to claim 30, wherein the control unit controls the dosage of UV-C by controlling the speed of the transportation means.

33. The apparatus according to claim 26, wherein the control unit controls the dosage of UV-C.

34. The apparatus according to claim 26, wherein the intensity of the UV-C is varied by increasing a number of germicidal UV-C lamps.

35. A method for removing one or more parts from a living plant said method comprising contacting the parts to be removed with a dosage of UV-C, wherein during a period of 24 hours, the dosage of UV-C is about 0.002 to about 1.0 J/cm² of tissue.

36. The method of claim 35 wherein at least one of the parts is a leaf or an aerial part.

37. A method of improving the vitality of a plant comprising exposing the plant to a dosage of UV-C, wherein during a period of 24 hours, the dosage of UV-C is about 0.002 to about 0.15 J/cm² of tissue.

38. The method of claim 37 wherein the vitality is improved by reducing the direct or indirect results of pathogen damage, or by causing the abscission of one or more parts of the plants.

39. The method of claim 37 wherein the vitality is improved relative to a comparable control plant that has not received a dosage of UV-C.
